(19) United States
(12) Patent Application Publication
(10) Pub. No.: US 2006/0073196 A1
(43) Pub. Date: Apr. 6, 2006

(54) Use of p-glycoprotein inhibitor surfactants at the surface of a colloidal carrier

(75) Inventors: Jean-Pierre Benoit, Avrille (FR); Alf Lamprecht, Gifu (JP)

Correspondence Address: FOLEY AND LARDNER LLP;SUITE 500, 3000 K STREET NW, WASHINGTON, DC, 20007, US

(73) Assignee: Institut National De La Sante Et De La Recherche Medicale (Inserm)

(21) Appl. No.: 10/545,238

(22) PCT Filed: Feb. 12, 2003
(86) PCT No.: PCT/IB03/00977

§ 371 Date: Aug. 12, 2005

(51) Int. Cl. A61K 31/704; A61K 31/4745; A61K 31/337; A61K 31/16; A61K 9/127

(52) U.S. Cl. 424/450; 514/34; 514/283; 514/449; 514/625

(57) ABSTRACT

Use of a colloidal carrier for the manufacture of a medicament for inhibiting P-glycoprotein, wherein said colloidal carrier:—encapsulates or adsorbs a pharmacologically active substance, and—comprises P-glycoprotein inhibitor surfactants bound to the colloidal carrier surface.

The subject matter of the present invention is related to the use of P-glycoprotein inhibitor surfactants at the surface of a colloidal carrier for inhibiting P-glycoprotein.

P-glycoprotein is a 170 KDa transmembrane protein member of the ABC family. Its normal role has been considered to be a detoxifying system in epithelial cells by stopping toxins or xenobiotics from entering into the cell. Its expression varies among different individuals which in turn is responsible for patient variability.

P-glycoprotein has been shown to act as an efflux pump and ejects many drugs (anticancer agents, antibiotics, antidepressants etc . . . ) from the cell in a similar way as bacterial transport proteins. The efficiency of many drugs is dramatically reduced by the P-glycoprotein efflux pump.

In particular, P-glycoprotein is well known as a factor contributing to the acquired multi-drug resistance syndrome (MDR) arising in many cancer patients after repeated chemotherapy [Kartner et al., 1985; Robinson et al., 1987]. Most of the anticancer drugs are affected by multidrug resistance.

Furthermore, a certain number of drugs such as the protease inhibitors used in the treatment of AIDS (Saquinavir®, Indinavir® . . . ) have a very weak bioavailability after oral administration. This is also explained by the presence of P-glycoprotein in the epithelium of the gastro-intestinal tract avoiding sufficient absorption due to the presence of the P-glycoprotein efflux pump. P-glycoprotein has been demonstrated to transport most HIV protease inhibitors (HPI) and to reduce their oral bioavailability and lymphocyte, brain, testis and fetal penetration, possibly resulting in major limiting effects on the therapeutic efficacy of these drugs [Huisman M T et al., 2002].

Although colloidal carriers could offer targeted delivery of drugs, thereby increasing efficiency and reduce adverse effects of drugs, this advantage was minimized when the targeted drug was affected by the multi-drug resistance phenomenon due to the presence of P-glycoprotein efflux pump.

PEG-HS (Solutol® HS 15 or polyethylene glycol-660 12-hydroxystearate) has been reported to inhibit the P-glycoprotein in cancer cells which causes the multi-drug resistance phenomenon [Buckingham et al., 1995; Buckingham et al., 1996].

All colloidal carriers preparation techniques are based either on phase separation or emulsification processes. A surfactant is generally required in all those techniques.

It was found recently that PEG-HS, which is a surfactant with amphiphilic properties, can be applied for the preparation of colloidal carriers.

In these cases, the surfactant is bound to the carrier surface by means of its lipophilic moiety. The hydrophilic polyethylene glycol chains of PEG-HS present at the carrier outer surface are stabilizing the carrier system in suspension. Furthermore, they induce a steric repulsion effect which minimizes the adhesion process of the carrier to the surface of macrophages and provides repulsive forces for the approaching plasma proteins. They therefore might allow avoiding an early uptake by the reticulo-endothelial system which is known from other opsonisation hindering systems.

It has now surprisingly been found that colloidal carriers containing P-glycoprotein inhibitor surfactants such as PEG-HS bound to their surface can release the drug into the aimed cell and also release said P-glycoprotein inhibitor surfactants.

Unexpectedly, the surfactants are not tightly bound to the carrier surface and are diffusing upon the presence of any kind of aqueous fluids. The P-glycoprotein inhibitor surfactants are thereby released into the aimed cell and can therefore inhibit the P-glycoprotein.

Those colloidal carriers which contain P-glycoprotein inhibitor surfactants bound at their surface are therefore capable of reducing the multi-drug resistance of cells. They are also capable of enhancing the oral bioavailability of drugs of which absorption by the epithelium is reduced by P-glycoprotein efflux pumps.

Those colloidal carriers have the advantage to deliver the drug to targeted cells and inhibit P-glycoprotein by the administration of one single delivery system.

The object of the present invention is the use of a colloidal carrier for the manufacture of a medicament for inhibiting P-glycoprotein, wherein said colloidal carrier:

encapsulates or adsorbes a pharmacologically active substance, and

comprises P-glycoprotein inhibitor surfactants bound to the colloidal carrier surface.

Such colloidal carrier allows the co-release of the pharmacologically active substance and of the P-glycoprotein inhibitor surfactants into the targeted cell. Advantageously, the pharmacologically active substance and the P-glycoprotein inhibitor surfactants are quasi-simultaneously released from the colloidal carrier into the targeted cell.

In one embodiment, the invention provides the use of such colloidal carrier to reduce multi-drug resistance of cells.

In a second embodiment, the invention provides the use of such colloidal carrier to enhance the oral bioavailability of a pharmacologically active substance of which absorption by the epithelium is reduced by the P-glycoprotein.

Colloidal carriers with P-glycoprotein inhibitor surfactants bound to their surface are predestinated for all applications in drug transport where a small carrier size is required to allow the site specific drug transport and which are impeded by biological efflux pumps.

One major example is the use in tumor treatment: hardly accessible tumor types such as glioblastoma can be targeted due to the small carrier size while inhibiting the P-glycoprotein efflux pump usually reducing the efficiency of the ordinary anticancer drugs.

Thus, such colloidal carriers can be used for drug delivery applications such as in oncology in order to target tumor cells and inhibit the multi-drug resistance simultaneously i.e. by the administration of one single drug delivery system.

Such colloidal carriers loaded with those drugs can inhibit the membrane protein related transporting systems and enhance the drug intracellular concentrations.

Such colloidal carriers further have the advantage that they do not need to be uptaken by the cell to release the drug into the cell.

Moreover, lots of P-glycoprotein inhibitor surfactants, such as PEG-HS, are already in use for injectable formulations and have been reported to have low cytotoxicity [Buckingham et al., 1995], while other P-glycoprotein inhibitors, e.g., nifedipine, which could be co-administered may cause severe adverse effects.

Methods for the preparation of colloidal carriers are well known from person skilled in the art.

According to the present invention, the colloidal carrier may be a nanoparticle such as a nanosphere, a nanocapsule, or a solid lipid nanoparticle, or it may be a liposome, a micelle, a nanosuspension, a nanoemulsion or a spherulite.

Nanoparticles may be defined as being submicronic (i.e. <1 μm) colloidal systems generally, but not necessarily made of polymers (biodegradable or not).

Nanoparticles include in particular nanospheres, nanocapsules and solid lipid nanoparticles.

Method for the preparation of nanoparticles including nanospheres and nanocapsules, are disclosed in Couvreur et al., Eur. J. Pharm. Biopharm, 41(1) 2-13, 1995.

Nanospheres are matrix systems in which the drug is dispersed throughout the particles.

Nanocapsules are systems in which the drug is confined to a cavity surrounded by a polymeric or a lipid membrane (Couvreur et al, Nanocapsule technology; Critical Reviews in Therapeutic Drug Carrier Systems, 2002). The size of nanocapsules is usually found to be between 80 and 500 nm. Nanocapsules are composed of a liquid core surrounded by a polymeric or a lipid membrane with lipophilic and/or hydrophilic surfactants at the interface. Nanocapsules allow parenteral administration (intravenous injection, intramuscular administration) or oral administration of drugs. Method for the preparation of polymeric nanocapsules is disclosed in P. Legrand et al., S.T.P. Pharma Sciences 9(5) 411-418, 1999.

In one embodiment of the invention, the colloidal carrier is a lipid nanocapsule prepared according to the process disclosed in WO 01/64328. The lipid nanocapsule prepared according to this process consists of an essentially lipid core that is liquid or semi-liquid at room temperature, coated with an essentially lipid film that is solid at room temperature. The average size of the nanocapsule is less than 150 nm, preferably less than 100 nm, more preferably less than 50 nm.

Solid lipid nanoparticles are nanospheres prepared from solid lipids such as triglycerides or fatty acids.

Methods for the preparation of solid lipid nanoparticles are disclosed in W. Mehnert, Advanced Drug Delivery Reviews 47(2001) 165-196.

Liposomes are spherical vesicles consisting of one or more phospholipid bilayers enclosing an aqueous phase. They can be classified as large multilamellar liposomes (MLV), small unilamellar vesicles (SUV) or large unilamellar vesicles (LUV), depending on their size and the number of lipid layers. Hydrophilic drugs can be solubilized in the inner aqueous core and lipophilic or amphiphilic compounds can be incorporeated into the lipid bilayers. Method for the preparation of liposomes are reviewed in Gregoriadis, G. (1993) Liposomes Technology (Vol. 1, 2^{nd} edn) CRC Press. Liposomes permit the intravenous injection or the oral administration of drugs.

Nanosuspensions are colloidal particles which are composed of the drug and the emulsifier only.

Micelles are surfactant aggregates that are able to entrap lipophilic molecules in an aqueous medium. They contain no aqueous core or lipid bilayers.

Nanoemulsions are sub micron emulsions. Methods for the preparation of nanoemulsions are disclosed in Osborne D W, Middleton Calif., Rogers R L, Alcohol free nanoemulsions, J. Disp. Sci. Technol., 9, 415-423, 1988.

Spherulites are concentric multilamellar microvesicles. Methods for the preparation of spherulites are disclosed in U.S. Pat. No. 5,792,472, U.S. Pat. No. 6,103,259 and in Freund et al., Life Sciences 67 (2000) 411-419 and Mignet et al., Nucleic Acid Research, Volume 28, Issue 16, 15 August 2000, pages 3134-3142.

According to the present invention the colloidal carrier is prepared from surfactants of which one at least is an inhibitor of the P-glycoprotein, i.e., which interacts with the P-glycoprotein thereby inactivating the P-glycoprotein efflux pumps.

Advantageously, surfactants which are inhibitor of the P-glycoprotein are amphiphilic. Furthermore they are non-ionic surfactants. More advantageously, they are fatty acid ester surfactants comprising a polyoxyethylene moiety, such as:
TPGS (polyethoxylated tocopheryl succinate)
Crémophor® EL (polyoxyethylene castor oil or polyethoxylated castor oil)
Tween® 20: polyoxyethylene sorbitan monolaurate
Tween® 40: polyoxyethylene sorbitan monopalmitate
Tween® 60: polyoxyethylene sorbitan monostearate
Tween® 80: polyoxyethylene sorbitan monooleate
Pluronic®P85 et L81 (polyoxyethylene-polyoxypropylene copolymers)
Triton X 100 (octylphenolethoxylate)
Nonidet P40 (Nonylphenyl polyoxyethyleneglycol)

Preferred P-glycoprotein inhibitor surfactants are:
Solutol® HS 15 (polyethylene glycol 660 12-hydroxystearate, Coon et al., 1991),
Cremophor® EL (polyoxyl 35 castor oil, Schuurhuis et al., 1990)
Tween® 80 (polyoxyethylene sorbitan monooleate, Riehm and Biedler, 1972).

According to the present invention, the surfactant is bound to the surface of the colloidal carrier, i.e. it can be anchored at the surface by means of its lipophilic moiety or adsorbed at the surface by means of weak chemical bounds.

According to the present invention, the pharmacologically active substance is encapsulated or adsorbed on the colloidal carrier.

“Encapsulated” means that the active substance is contained inside the colloidal carrier.

“Adsorbed” means that the active substance is adsorbed at the outer surface of the colloidal carrier.

The pharmacologically active substance encapsulated in the colloidal carrier according to the present invention may be any pharmacologically active substance which undergoes ejection from the cells by the P-glycoprotein efflux pumps. Advantageously, it may be any drug which undergoes multidrug resistance. More advantageously, it may be any anti-cancer drug which undergoes multidrug resistance.

Accordingly, the pharmacologically active substance may be an anti-cancer drug such as vinblastine, colchicines, paclitaxel, etoposide, docétaxel, vincristine or téniposide.

Accordingly, the cells which are targeted by the colloidal carrier according to the present invention may be tumor cells, such as glioblastoma, liver metastasis, colorectal cancer cells, lung cancer cells, myeloma, prostate cancer cells, breast cancer cells or ovarian cancer cells.

The very weak availability of many drugs after oral administration has been explained by the presence of the P-glycoprotein in the epithelium of the gastro-intestinal tract, thereby avoiding sufficient absorption of the drug by the epithelium.

Accordingly, the pharmacologically active substance may be a protease inhibitor. As such, it may be a drug for treating AIDS such as Saquinavir® or Indinavir®.

The pharmacologically active substance may also be an antibiotic drug such as azithromycin, clarithromycin, erythromycin, roxithromycin, dirithromycin, clindamycin, dalfopristin and tetracycline.

In all the following description:
P-gp=P-glycoprotein
PEG-HS=Solutol® HS 15 or polyethylene glycol 660 12-hydroxystearate (Coon et al., 1991),
LNC=lipid nanocapsules prepared according to the process disclosed in WO 01/64328
Blank LNC=unloaded lipid nanocapsules i.e. LNC which does not encapsulate any pharmacologically active substance
Loaded LNC=lipid nanocapsules which do encapsulate a pharmacologically active substance
SPIbio® test system=P-gp drug interaction assay kit manufactured by SPIbio, Massy, France.
SD=standard deviation
F98 cell=CRL-2397
9L cell=ECACC 94110705
PX=paclitaxel

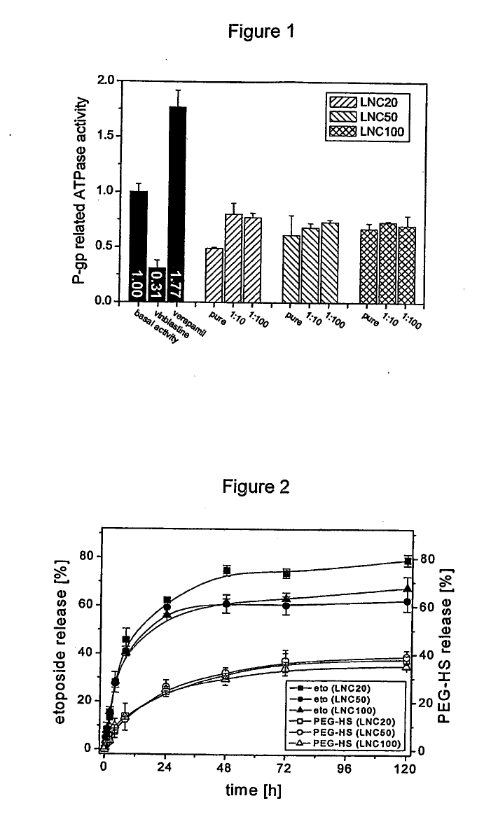

FIG. 1: P-gp interaction experiments with the SPIbio® test system for the different LNC formulations at varying carrier concentrations.

FIG. 2: Release profiles of different etoposide loaded LNC formulations in a phosphate buffer release medium at pH 7.4 and 37° C.

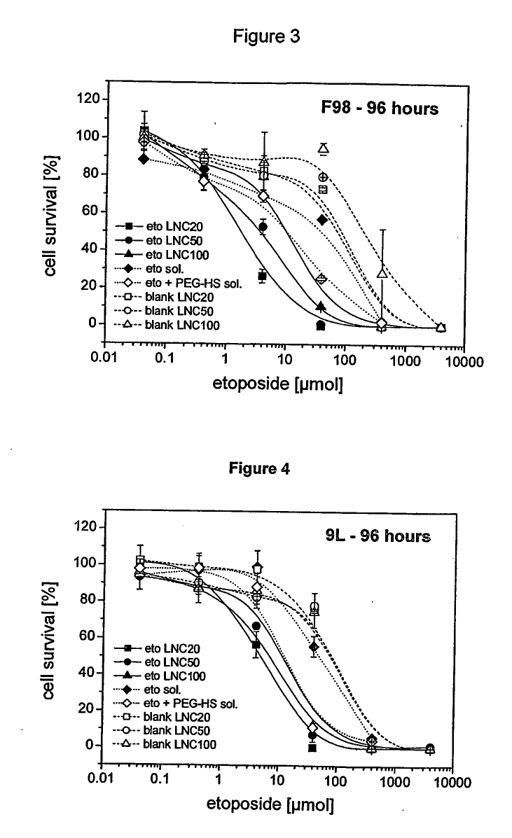

FIG. 3: Etoposide loaded LNC of different batches compared with equivalent blank LNC or etoposide solution of similar concentration in F98 cells.

FIG. 4: Etoposide loaded LNC of different batches compared with equivalent blank LNC or etoposide solution of similar concentration in 9L cells.

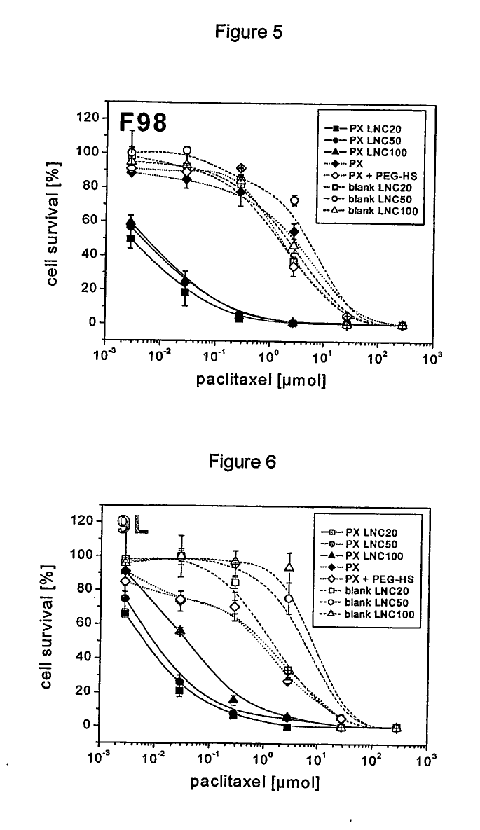

FIG. 5: Paclitaxel loaded LNC of different batches compared with equivalent blank LNC or PX solution of similar concentration in F98 cells.

FIG. 6: Paclitaxel loaded LNC of different batches compared with equivalent blank LNC or PX solution of similar concentration in 9L cells.

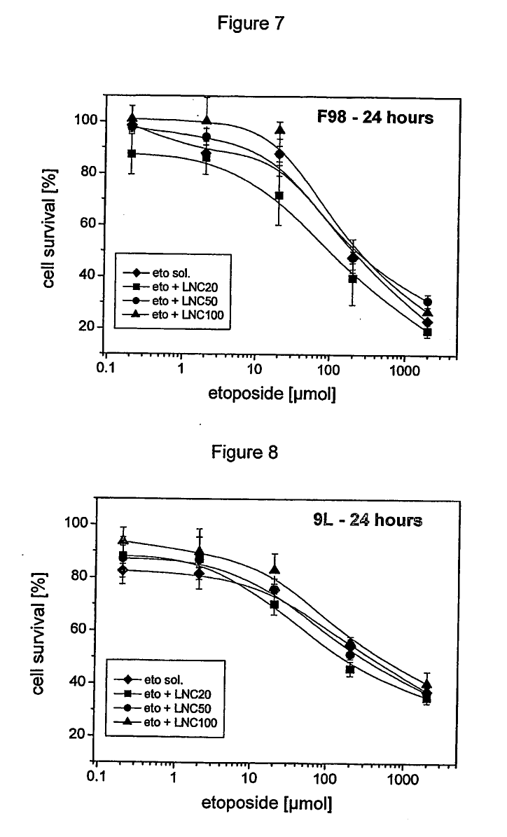

FIG. 7: Etoposide solution pure or combined with blank LNC of different batches in F98 cells.

FIG. 8: Etoposide solution pure or combined with blank LNC of different batches in 9L cells.

### EXAMPLES

Materials and Methods

Nanocapsule Preparation

Lipid nanocapsules are prepared according to the process disclosed in WO01/64328.

The drug was dissolved in neutral oil by ultrasonication prior to all the preparation steps.

Thereafter, the different LNC formulations at nominal sizes of 20, 50, and 100 nm were based on the new preparation method of phase inversion processing recently reported in literature [Heurtault et al., 2001].

Briefly, all components (phosphatidylcholine, PEG-HS, sodium chloride, triglycerides, and water) at their various concentrations were mixed and heated under magnetic stirring up to 85° C. in order to ensure to pass the phase inversion temperature. The following cooling step was performed until a temperature of 55° C. passing back the phase inversion zone completely again. This cycle was repeated another two times before adding 5 ml of distilled water at 2° C. The formulation was stirred for another 10 minutes before further use.

Determination of Drug Release Kinetics and PEG-HS Release

The in-vitro release kinetics of the LNC were performed by a dialysis method since centrifugation did not allow the separation of the LNC in an adequate time interval due to their small diameter. 3 ml of drug-loaded LNC suspension was filled into a dialysis tube and inserted in a 100 ml flask containing a phosphate buffer (pH 7.4) in a water bath at 37° C. under gentle magnetic stirring at 250 rpm. At appropriate intervals, 0.5 ml samples were withdrawn and assayed for drug release and replaced by 0.5 ml of fresh buffer. The amount of drug in the release medium was determined by high performance liquid chromatography (HPLC).

For the PEG-HS release, 1 ml of carrier suspension was filled into a dialysis tube and inserted in a 100 ml flask containing a phosphate buffer (pH 7.4) in a water bath at 37° C. under gentle magnetic stirring at 250 rpm. At appropriate intervals, 0.5 ml samples were withdrawn and assayed for free PEG-HS in the phosphate buffer. The quantification of PEG-HS was performed by a color reaction with potassium iodide and UVNIS detection at 500 nm [McAllister and Lisk, 1951].

P-Glycoprotein Interaction Experiments

Different batches of Blank LNC were prepared varying LNC size and varying dilutions of suspension of LNC in water.

Pure LNC

| | | Solid excipients concentration | |
|---|---|---|---|
| | | (100% of the excipients | |
| LNC size | | are within the LNC) | Number of LNC |
| 20 | nm | 0.175 g/ml solids | 7.71 × 10¹⁵ LNC/ml |
| 50 | nm | 0.194 g/ml solids | 7.68 × 10¹⁴ LNC/ml |
| 100 | nm | 0.288 g/ml solids | 1.54 × 10¹⁴ LNC/ml |

```
Varying dilution of the suspensions
1:1
  1:10
  1:100
```

Blank LNC of the different batches were applied to the P-gp drug interaction assay kit (SPIbio®, Massy, France) in order to determine some type of interaction between the LNC and the membrane located P-glycoprotein.

The commercially available test was performed according to the supplier's instructions. Briefly, in 96 well plates the different LNC formulations were incubated with the P-gp exhibiting membrane vesicles for 20 min at varying concentrations. All measurements were based on the ATPase activity of P-gp which was linked to an enzymatic cascade of pyruvate kinase and lactate dehydrogenase where NADH was quantified in UV at 340 nm [Garrigues et al., 2000].

Prior to these experiments, the membrane vesicles were tested for their stability in the presence of the LNC.

Cell Culture

Glioma cell lines of F98 and 9L were obtained from ATCC (Manassas, Va., USA). Approximately 10.000 cells per well were seeded in 24 cavity well plates with a poly-D-lysine coating and grown in DMEM.

Thereafter, cells were incubated with either drug solution or blank LNC or drug loaded LNC of equivalent drug or excipient concentration. In order to prevent a misleading positive effect by the toxicity of any of the used capsule components, excipients were applied in equivalent quantities. This is, surely, equivalent to the PEG-HS concentration.

The incubation periods were 96 hours for the appropriate formulations.

All formulations were containing 2 to 2000 micromole/ml of drug.

Blank LNC contained equivalent masses of excipients compared to drug-loaded LNC.

The cell survival after the treatment period was tested with the MTT test [Carmichael et al., 1987].

Cytotoxicity was expressed as percentage of controls (untreated cells).

A primary rat cell culture of astrocytes was grown in 24er wells for about 3 weeks. Oligodendrocytes were removed and then the confluent cells were used in cytotoxicity tests by applying carrier formulations or free drug at equivalent concentrations.

Results and Discussion

P-gp Inhibition

Results are shown at FIG. 1.

All results are shown as mean±SD for four measurements.

The basal activity of P-gp depending ATP-ase of the test system itself was taken as 1.0 value.

Comparable standards are given by the basal activity of the test system with additional results from vinblastine and verapamil.

The tested LNC samples showed a decrease of the relative P-gp activity expressed in ATPase activity. A likely origin of this phenomenon is a P-gp inhibition.

In the SPIbio® test system, a slightly LNC size dependent P-gp inhibition was observed.

Pure LNC suspensions were found to lower significantly the P-gp related ATP-ase activity for all capsule sizes.

In all batches the inhibition slightly varied with the different concentrations where this influence on the ATP-ase activity was found only to be significant for LNC20 formulations. This proved P-gp related ATP-ase inhibition might be essentially based on the activity of free PEG-HS which has been reported in literature to be an efficient P-gp inhibitor to the multidrug resistance phenomenon.

The surfactant is not tightly bound to the LNC surface and is diffusing upon the presence of any kind of aqueous fluid. From this point of view, LNC can be seen as a reservoir for the incorporated drug and simultaneously its P-gp inhibiting surfactant PEG-HS. A combined delivery of both, drug and P-gp inhibitor, into the aimed tissue might permit to increase enormously the efficiency of such a system.

LNC Properties and Drug and Pgp-Inhibitor Release In-Vitro

An example for the in-vitro drug release kinetics obtained from etoposide by representing the percentage of cumulated drug release in phosphate buffer (pH 7.4 at 37° C.) for different LNC formulations is shown in FIG. 2.
Eto(LNC20, LNC50, LNC100)=weight percentage of etoposide released from 20 nm, 50 nm, 100 nm LNC loaded with etoposide.
PEG-HS(LNC20, LNC50, LNC100)=weight percentage of PEG-HS released from 20 nm, 50 nm, 100 nm LNC loaded with etoposide

It can be clearly seen from this example that there is a dual release taking place, one of the anticancer drug and the one of the P-gp inhibitor PEG-HS.

About 35% of the initial surfactant mass is released which represents a significant amount for the P-gp inhibition at the site of action. Moreover, the stability of the carrier system is not impeded by the surfactant dislocation.

Cell Culture Experiments

A) Results shown at FIGS. 3 and 4 (etoposide) and FIGS. 5 and 6 (paclitaxel).

Eto (LNC20, LNC50, LNC100)=20 nm, 50 nm, 100 nm LNC loaded with etoposide.
Eto sol.=etoposide solution
Eto+PEG-HS sol.=free administred etoposide and PEG-HS solution (PEG-HS concentration was equivalent to the 35% values of the corresponding LNC formulation, which is freely available after the release from the LNC over 48 hours), see FIG. 2.
Blank (LNC20, LNC50, LNC100)=unloaded 20 nm, 50 nm, 100 nm LNC PX (LNC20, LNC50, LNC100)=20 nm, 50 nm, 100 nm LNC loaded with paclitaxel
PX=paclitaxel solution
PX+PEG-HS sol.=paclitaxel solution and PEG-HS solution (PEG-HS concentration was equivalent to the 35% values of the corresponding LNC formulation, which is freely available after the release from the LNC over 48 hours), see FIG. 2.

A distinct difference in efficiency was found between drug solution and drug loaded LNC for all tested cell lines after 96 hours incubation period.

The IC₅₀ values of cell growth inhibition for etoposide varied from a 25 fold higher inhibition of cell growth for F98 to at least a 8 fold increase of efficiency in 9L cells.

In the case of paclitaxel, the effect and the differences were much more dramatic. The effect of paclitaxel loaded LNC on F98 cells was around 300 times higher than the pure drug while the inhibition on the cell growth in 9L cells was still 80 fold higher than the drug.

For the different LNC diameters, a size dependent strength of the effect was observed. Blank LNC proved a cytotoxic value always lower than the free administered drug solution and a high toxicity of the excipients could be excluded.

B) Results Shown at FIGS. 7 and 8

In order to prove the reservoir theory of the additional effect of the presence of LNC, cells were incubated with free drug in the presence of blank LNC at a non-toxic concentration (1:1000 dilution).

In this case F98 exhibited a higher sensitivity for such a treatment (FIG. 7).

Moreover, IC₅₀ values showed an influence of the LNC diameter on this effect where for all cell lines the smallest LNC were found to be the most efficient This might be based on the presence of a higher absolute amount of free PEG-HS (according to FIG. 2) increasing the P-gp inhibiting effect. Such results are in line with the hypothesis of a P-gp dependent efficiency of the LNC system.

Compared to 9L and F98 cell growth was found to be affected to a higher extent by the combination drug/blank LNC, probably mainly based on the inhibition of their multidrug resistance mechanism [Matsumoto et al. 1992].

9L cells are reported in the literature to show only little P-gp expression and have to be expected to show a lower P-gp depending multi-drug resistance (Saito et al., 1991; Yamashima et al., 1993).

In general, it seems that the mode of action of nanocarriers on cancer cells is combining two different pathways.

An enhanced cell death can occur by blocking the P-gp depending efflux pumps with PEG-HS and subsequently increasing the drug concentration inside the cytoplasm. This hypothesis was supported by the experiments shown in FIG. 7, where blank LNC combined with free drug displayed a distinct effect on and F98 cells, but not in 9L. This higher efficiency in the presence of drug-free LNC speaks for the P-gp blocking mechanism, especially since differences were significantly lower in 9L cells which are reported to have less or no P-gp expression. However, in the 9L experiments LNC still show a up to 8-fold higher efficiency than the free drug. Thus, an antitumor activity may also be reached by a second pathway. Due to the very reduced size of these nanocarriers a distinct uptake into the cells occurred. This intracellular presence of the drug carrier may also be able to circumvent the multidrug resistance mechanisms as also reported from earlier work [Bennis et al., 1994; Hu et al., 1995].

When equivalent doses of LNC were applied to rat astrocytes in primary cell culture they were found only to have a minimally higher toxicity compared with free PX. These observations were similar for blank or PX loaded LNC. Such findings support the innocuousness of this new treatment method.

### CONCLUSIONS

The previously described nanocarrier system allows a combined release of anticancer drug and P-gp inhibitor from the same system, which is in favor of an application against the multi-drug resistance in cancer. After the entrapment of an anticancer drug, the new strategy was found to inhibit glioma cell growth, in some cases LNC were more than 20 fold more efficient than the drug in solution. The unique advantage of this system is the controlled delivery of both, drug and inhibitor at the same time where the inhibitor is of lowered toxicity and does not require the administration of an additional component.

### REFERENCES

Heurtault B, Saulnier P, Pech B, Proust J E, Benoit J P. A Novel Phase Inversion-Based Process for the Preparation of Original Lipid Nanocarriers. Pharm Res, 2002, 19, 875-880.
Bennis S, Chapey C, Couvreur P, Robert J. Enhanced cytotoxicity of doxorubicin encapsulated in polyisohexylcyanoacrylate nanospheres against multidrug-resistant tumour cells in culture. Eur J Cancer. 1994; 30A(1):89-93.
Hu Y P, Henry-Toulmé N, Robert J. Failure of liposomal encapsulation of doxorubicin to circumvent multidrug resistance in an in vitro model of rat glioblastoma cells. Eur. J. Cancer. 1995; 3: 389-394.
Olivier J C, Fenart L, Chauvet R, Pariat C, Cecchelli R, Couet W. Indirect evidence that drug brain targeting using polysorbate 80-coated polybutylcyanoacrylate nanoparticles is related to toxicity. Pharm Res. 1999 December; 16(12):183642.
Fernandez-Urrusuno R, Fattal E, Porquet D, Feger J, Couvreur P. Evaluation of liver toxicological effects induced by polyalkylcyanoacrylate nanoparticles. Toxicol Appl Pharmacol. 1995 February; 130(2):272-9.
Matsumoto Y, Sasaoka N, Tsuchida T, Fujiwara T, Nagao S, Ohmoto T. Fluorescent dye rhodamine 6G as a molecular probe to study drug resistance of C6 rat glioma cells. J Neurooncol. 1992 July; 13(3):217-22.
Carmichael J. DeGraff W G, Gazdar A F, Minna J D, Mitchell J B. Evaluation of a tetrazolium-based semiautomated colorimetric assay: assessment of chemosensitivity testing. Cancer Res. 1987 Feb. 15; 47(4):936-42.
Garrigues, J. Nugier, J. Ferté, S. Orlowski, E. Ezan. In Vitro Drug Screening Assay for Testing Interaction with P-Glycoprotein Based on its ATPase Activity, 2000 MPS ANNUAL MEETING, Indianapolis, USA.
McAllister R V, Lisk, R J, Polyoxyethylene stearate—colorimetric determination in dilute solutions. Anal. Chem., 1951, 23:609-610.
Couvreur P., Barraft G., Faftal E., Legrand P., Vauthier C., Nanocapsule technology: a review. Critical Reviews in Therapeutic Drug Carrier Systems, 2002, 19(2), 99-134.
Saito Y, Nakada Y, Hotta T, Mikami T, Kurisu K, Yamada K, Kiya K, Kawamoto K, Uozumi T. Cross-resistance patterns in ACNU-resistant glioma sublines in culture. J Neurosurg 1991 August; 75(2):277-283.
Yamashima, T., Ohnishi T, Nakajima Y, Terasaki T, Tanaka M, Yamashita J, Sasaki T, Tsuji A. Uptake of drugs and expression of P-glycoprotein in the rat 9L glioma. Exp. Brain Res., 1993, 95, 41-50.
Kartner N., Evemden-Porelle D. and Ling V., Detection of P-glycoprotein in multidrug resistant cell lines by monoclonal antibodies. Nature, 316(1985) 820-823.
Robinson I. B., Molecular mechanism of multidrug resistance in tumor cells. Clin. Physiol. Biochem., 5(1987) 140-151.
Huisman M T, Smit J W, Crommentuyn K M, Zelcer N, Wiltshire H R, Beijnen J H, Schinkel A H, Myultidrug resistance protein 2 (MRP2) transports HIV protease inhibitors, and transport can be enhanced by other drugs, AIDS 2002 Nov. 22; 16(17):2295-2301.

1. Use of colloidal carrier for the manufacture of a medicament of inhibiting P-glycoprotein, wherein said colloidal carrier:
encapsulates or adsorbes a pharmacologically active substance, and
comprises P-glycoprotein inhibitor surfactants bound to the colloidal carrier surface.

2. Use of a colloidal carrier according to claim 1, said colloidal carrier allowing the pharmacologically active substance and the P-glycoprotein inhibitor surfactants to be co-released into the targeted cell.

3-13. (canceled)
